(19) United States
(12) Patent Application Publication
(10) Pub. No.: US 2018/0144522 A1
(43) Pub. Date: May 24, 2018

(54) IMAGE POSITIONING AND STITCHING METHOD AND IMAGE DETECTION SYSTEM OF CELL DETECTION CHIP

(71) Applicant: National Tsing Hua University, Hsinchu City (TW)

(72) Inventors: Yi-Hsuan Weng, Hsinchu City (TW); Yu-Chen Cheng, Hsinchu City (TW); Fan-Gang Tseng, Hsinchu City (TW)

(73) Assignee: National Tsing Hua University, Hsinchu City (TW)

(21) Appl. No.: 15/436,438

(22) Filed: Feb. 17, 2017

(30) Foreign Application Priority Data
Nov. 21, 2016 (TW) 105138138

(51) Int. Cl. G06T 11/60; G06T 7/38; G01N 33/574

(52) U.S. Cl. CPC G06T 11/60; G01N 33/574; G06T 7/38

(57) ABSTRACT

An image positioning and stitching method and an image detection system of a cell detection chip are provided. In the method, a moving path of an image capturing device for capturing images of the cell detection chip is planned according to a size of an imaging region of the image capturing device and a size of a detection region of the cell detection chip, wherein a plurality of marks for positioning the images are disposed in the detection region. Next, the image capturing device is controlled to move above the cell detection chip according to the planned moving path to capture a plurality of images of the cell detection chip. Lastly, the images are positioned and stitched into a complete chip image according to the positions of the marks appearing in the images.

### CROSS-REFERENCE TO RELATED APPLICATION

This application claims the priority benefit of Taiwan application serial no. 105138138, filed on Nov. 21, 2016. The entirety of the above-mentioned patent application is hereby incorporated by reference herein and made a part of this specification.

### BACKGROUND OF THE INVENTION

### Field of the Invention

The invention relates to a cell detection method and device, and more particularly, to an image positioning and stitching method and an image detection system of a cell detection chip.

### Description of Related Art

With the development of the biotechnology industry, the method of detecting cancer cells has also gradually been developed. One of the detection methods is circulating tumor cell (CTC) detection, i.e., detecting cancer cells in the circulatory system. Although the current CTC analysis is still in the research stage, analysis of CTC numbers still has its clinical value. For instance, a doctor can predict tumor metastasis and evaluate treatment methods via the number of CTC in the patient's blood. The quantity of the CTC number is generally related to the possibility of tumor metastasis, and therefore a greater number thereof means a higher chance of metastasis, which also represents the extent of tumor growth.

When a specimen of blood is obtained, since the number of cells in the sample is relatively large, in particular red blood cells, etc., a purification step needs to be first performed. The CTC separation technology is mainly divided into four types, which are respectively cell density gradient centrifugation, cell size sorting (similar to the concept of filtering with a filter), capture of immune antibody by a microstructure, and immunomagnetic beads separation. After CTC is isolated by different methods, analysis of, for instance, DNA and fluorescent signal is performed. In CTC separation, since the quantity of the blood sample is large, high-throughput biochips are needed to detect large amounts of cells at the same time, such as chips arranged in a two-dimensional array, and the arrangement range of the cells is limited by a 5-micrometer gap generated at the edge of the chips to prevent cell loss. Since the two-dimensional array can be arranged into a single-layer structure, high-sensitivity detection can be achieved using the contrast of cell fluorescence.

However, even if the structure of the chip is simplified and the operating time of the chip is reduced as a result, a relatively long time is still needed for the detection.

### SUMMARY OF THE INVENTION

The invention provides an image positioning and stitching method and an image detection system of a cell detection chip that can provide a positioning stitching function of the captured chip image.

The image positioning and stitching method of a cell detection chip of the invention is suitable for an electronic device having a processor to control an image capturing device to capture images of the cell detection chip and position and stitch the captured images. In the method, a moving path of the image capturing device capturing the images of the cell detection chip is planned according to a size of an imaging region of the image capturing device and a size of a detection region of the cell detection chip, wherein a plurality of marks for positioning the images are disposed in the detection region. Next, the image capturing device is controlled to move above the cell detection chip according to the planned moving path to capture a plurality of images of the cell detection chip. Then, the images are positioned and stitched into a complete chip image according to the positions of the marks appearing in the images.

In an embodiment of the invention, the step of positioning and stitching the images into a complete chip image according to the positions of the marks appearing in the images further includes finding adjacent images according to an order of image capture or a position of image capture in the moving path of each of the images and stitching the adjacent images according to the positions of the marks appearing in the adjacent images to obtain the complete chip image.

In an embodiment of the invention, the number and the position of the marks in the cell detection chip include deciding according to a size of the imaging region of the image capturing device and a size of the detection region of the cell detection chip such that each of the images captured by the image capturing device includes at least one of the marks.

In an embodiment of the invention, the method further includes producing the marks using a photoresist on the cell detection chip, wherein the photoresist includes SU-8 or AZ9260.

In an embodiment of the invention, the step of producing the marks using a photoresist on the cell detection chip includes coating a first photoresist on a slide surface and performing soft bake, exposure, and development to produce a microstructure of a runner and coating a second photoresist at the position of each of the marks on the slide surface and perform soft bake, exposure, and development to produce a microstructure of the mark.

The image detection system of a cell detection chip of the invention includes an image capturing device and an electronic device. In particular, the image capturing device is disposed above the cell detection chip and is moved to capture a plurality of images of the cell detection chip. The electronic device includes a connection device, a storage device, and a processor. The connection device is used to connect the image capturing device. The storage device is used to store a plurality of modules. The processor is coupled to the connection device and the storage device to execute the modules stored in the storage device. The modules include a moving path planning module, a control module, and an image stitching module. The moving path planning module plans the moving path of the image capturing device capturing the images of the cell detection chip according to the size of the imaging region of the image capturing device and the size of the detection region of the cell detection chip, wherein a plurality of marks for positioning the images is disposed in the detection region. The control module is used to control the image capturing device to move above the cell detection chip according to the moving path planned by the moving path planning module to capture the images. The image stitching module is used to position and stitch the images into a complete chip image according to the positions of the marks appearing in the images.

In an embodiment of the invention, the image stitching module includes finding adjacent images according to an order of image capture or a position of image capture in the moving path of each of the images and stitching the adjacent images according to the positions of the marks appearing in the adjacent images to obtain the complete chip image.

In an embodiment of the invention, the moving path planning module further decides a number and position of the marks in the cell detection chip according to the size of the imaging region of the image capturing device and the size of the detection region of the cell detection chip such that each of the images of the image capturing device includes at least one mark.

In an embodiment of the invention, the marks include one of numbers, alphabets, signs, and patterns or a combination thereof.

Based on the above, in the image positioning and stitching method and the image detection system of a cell detection chip of the invention, images of each of the regions in a detection region of the cell detection chip which are disposed with marks for positioning are captured by a camera in a manner of recording and timing capture. Next, via an image stitching algorithm, the marks in each of the images and images having the same marks are found, and then each of the images is positioned and stitched according to the positions of the marks in the images such that the marks in the images are overlapped after stitching. Via the assistance of the marks, the electronic device can rapidly identify the overlapped portions and stitch the chip images into a complete chip image.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A and FIG. 1B are respectively schematic diagrams illustrating a cell detection platform and a cell detection chip according to an embodiment of the invention.

FIG. 2 is a block diagram of the image detection system of a cell detection chip according to an embodiment of the invention.

FIG. 3 is a flow chart illustrating the image positioning and stitching method of a cell detection chip according to an embodiment of the invention.

FIG. 4 is a schematic diagram illustrating the detection region of a cell detection chip according to an embodiment of the invention.

FIGS. 5A to 5E are schematic diagrams of the produce of a runner and a mark microstructure shown according to an embodiment of the invention.

FIG. 6A is a schematic diagram illustrating the movement of an image capturing device according to an embodiment of the invention.

FIG. 6B and FIG. 6C are schematic diagrams illustrating image stitching according to an embodiment of the invention.

FIG. 7A and FIG. 7B are schematic diagrams illustrating image stitching according to the positions of marks in the images according to an embodiment of the invention.

### DESCRIPTION OF THE EMBODIMENTS

In an embodiment of the invention, circulating tumor cell (CTC) and lymphocytes, etc., are directly isolated from blood using density centrifugation (such as Ficoll) and fluorescence calibration is performed on the isolated samples, and then the samples are added dropwise into a self-assembled cell array (SACA) chip of an embodiment of the invention. The chip allows the samples added dropwise to show a cell monolayer arrangement after a period of time via fluid properties.

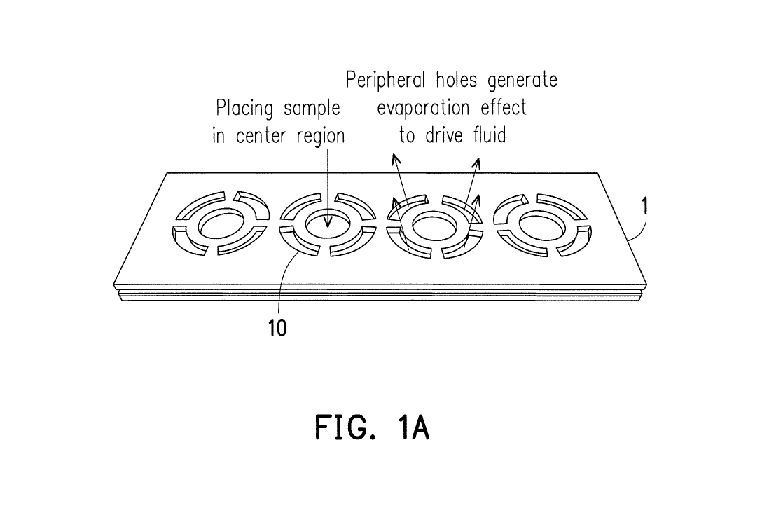

Specifically, in an embodiment of the invention, cell sorting is implemented using a micro channel well testing platform and cell placement can be dispersed via fluid action to prevent an excess number of cells in the same placement causing lowered detection effect. FIG. 1A and FIG. 1B are respectively schematic diagrams illustrating a cell detection platform and a cell detection chip according to an embodiment of the invention. Referring to FIG. 1A, a plurality of cell detection chips (such as a chip 10) can be disposed on a cell detection platform 1, a transparent material (such as a slide), etc. is used as the platform body, and a runner about 5 μm thick is formed on the slide surface via photolithography as the power source of cell flow. Specifically, the round hole in the middle of the chip 10 is used as the injection hole of the cell suspension, and the holes in the periphery of the chip 10 are used as evaporation holes such that the liquid in the runner can be evaporated. The outward pulling from liquid evaporation not only can provide a stable pulling force to the flow of the cells to spread the cells in the center outward, but can also prevent a high water evaporation rate causing cell death.

Referring to FIG. 1B, the chip 10 is, for instance, formed by stacking two slides 12 and 14 on top of each other, and a layer of a photoresist 16 of 5 μm thick formed by photolithography is disposed between the two slides 12 and 14. The opening in the middle of the slide 12 is used as an injection hole of cell suspension, and the thickness of the photoresist 16 is used as a wall of the well, such that the bottom of the well has a narrow channel for outward flow, and the liquid in the well is evaporated to the outside via the peripheral holes by passing through the runner. Two forces are caused by the evaporation: one is lateral pulling that can cause cells in the center of the well to move outward and generate a “leveling” phenomenon, and the other is downward pulling that can accelerate the settlement and arrangement of cells. Via a slight acceleration of arrangement, the possibility of cell accumulation can be reduced.

It should be mentioned that, to prevent loss of cells with the evaporation of the liquid in the well, the thickness of the photoresist 36 is set to at least 5 micrometers such that cells having a diameter greater than the thickness are confined in the well. It can be known from the enlarged drawing on the right side of FIG. 1B that, the function of the photoresist 36 is similar to a filter hole that allows liquid to pass through, but the cells are stopped by the two vertically-stacked slides 12 and 14 and remain in the well.

In the following, the process of injecting the cells in the chip 10 is briefly described with reference to FIG. 1B. First, a PBS solution is filled in the chip 10, and then a cell suspension 18 is added dropwise from the cell suspension injection hole. At this point, a cell 18a is affected by the downward gravity and lateral pulling, and cells closer to the edge of the well rapidly flow toward the lateral runner at the bottom due to the influence of the lateral pulling, and lastly the cells are blocked by the narrow filter holes and confined in the well. As a result, excessive dispersion of cells can be avoided. Moreover, although the lateral pulling to cells close to the center is less, the cells still settle downward due to gravity, and lastly cells settled at the same position are leveled (such as a cell 18b), and therefore failing to form a single-layer arrangement due to the stacking of cells would not be occurred.

In order to complete cell detection in the chip 10 in a short period of time, in an embodiment of the invention, an automatic detection imaging system is designed for the chip 10, wherein the system reserves a mark in the detection region of the chip 10 and controls the camera to move on the chip 10 via an electronic control platform to record chip images, and then performs positioning and stitching on the captured images using the marks in the captured images to obtain a complete chip image. Accordingly, in addition to significantly reducing the time for manual operation, researchers also do not need to spend effort for the subsequent image analysis.

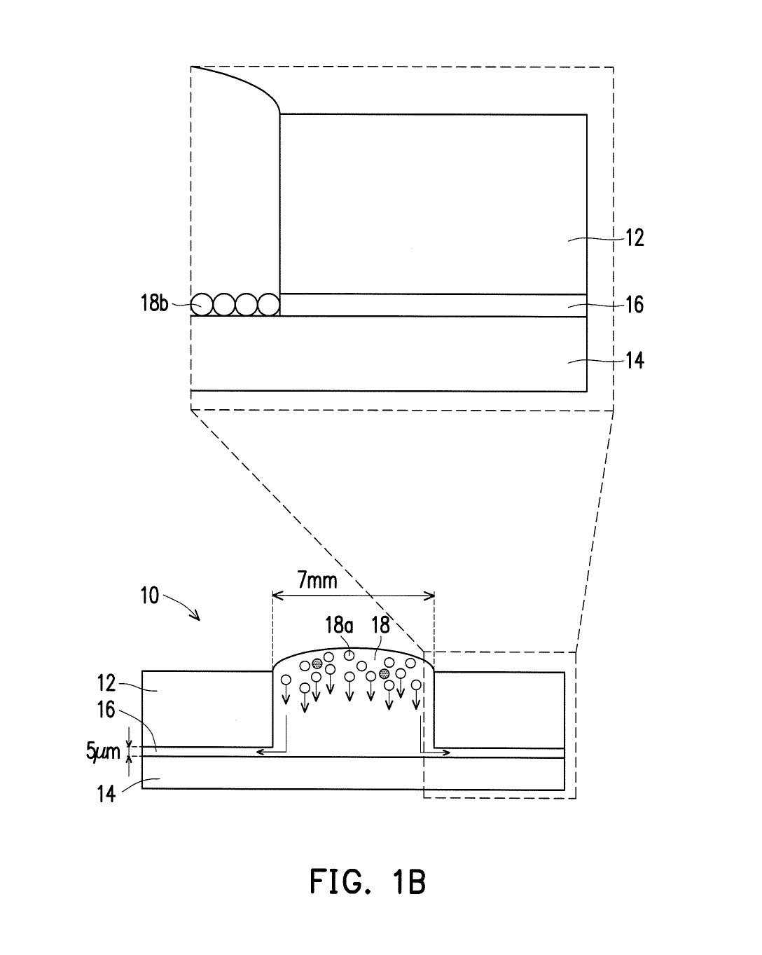

For instance, FIG. 2 is a block diagram of the image detection system of a cell detection chip according to an embodiment of the invention. Referring to FIG. 2, an image detection system 2 of the present embodiment includes an electronic device 20 and an image capturing device 30, wherein the electronic device 20 is, for instance, a PC, a server, a workstation, or a calculator device having computing capability and includes a connection device 22, a storage device 24, and a processor 26. The functions thereof are described below.

The connection device 22 is, for instance, a universal serial bus (USB), an RS232 interface, a universal asynchronous receiver and transmitter (UART), an integrated circuit bus (I2C), a serial peripheral interface (SPI), a display port, a Thunderbolt interface, or a local area network (LAN) interface that allows the electronic device 20 to be connected to the image capturing device 30 in a wired manner to control the image capturing device 30 to move and capture images.

It should be mentioned that, the image capturing device 30 includes, for instance, a component such as a lens, an image sensor, and an actuator. The lens is, for instance, formed by one or a combination of a plurality of a concave-convex lens, and the position of the lens is changed to change the focal length so as to focus on the object to be photographed. The image sensor includes, for instance, a charge-coupled device (CCD), a complementary metal-oxide semiconductor (CMOS) device, or other types of photosensitive devices that can sense the light intensity entering the lens to generate an image. The actuator is, for instance, a stepper motor that can drive the lens of the image capturing device 30 to move above the cell detection chip according to the control signal emitted by the electronic device 20 so as to capture images of the cell detection chip.

The storage device 24 can be any type of fixed or movable random access memory (RAM), read-only memory (ROM), flash memory, a similar device, or a combination of the devices. In the present embodiment, the storage device 24 is used to store a moving path planning module 242, a control module 244, and an image stitching module 246, and these modules store, for instance, programs in the storage device 24.

The processor 26 is, for instance, a central processing unit (CPU) or other programmable microprocessors for conventional use or special use, a digital signal processor (DSP), a programmable controller, an application specific integrated circuit (ASIC), a programmable logic device (PLD), other similar devices, or a combination of the devices. The processor 26 is coupled to the connection device 22 and the storage device 24 and loads the programs of the moving path planning module 242, the control module 244, and the image stitching module 246 from the storage device 24 to execute the image positioning and stitching method of a cell detection chip of the invention. In the following, embodiments are provided to describe the detailed steps of the method.

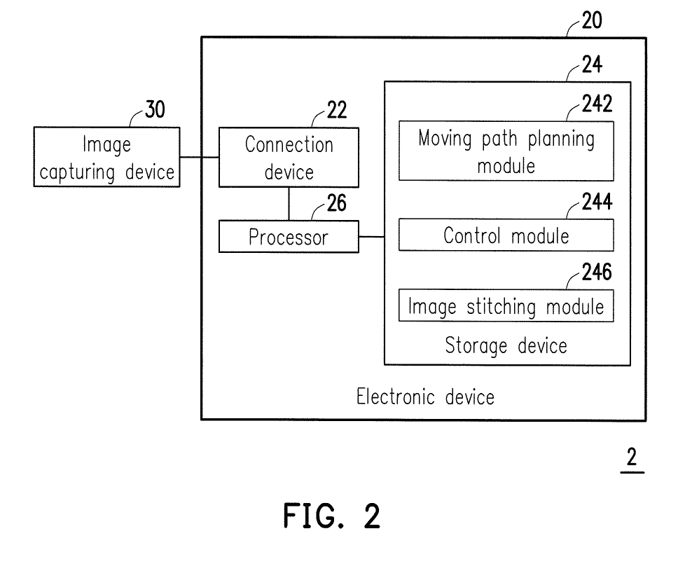

FIG. 3 is a flow chart illustrating the image positioning and stitching method of a cell detection chip according to an embodiment of the invention. Referring to both FIG. 2 and FIG. 3, the method of the present embodiment is suitable for the image detection system 2 of FIG. 2, and in the following, the detailed steps of the image positioning and stitching method of the invention are described with reference to each of the components in the image detection system 2.

First, the processor 26 of the electronic device 20 executes the moving path planning module 242 to plan the moving path of the image capturing device 30 for capturing images of the cell detection chip according to a size of an imaging region of the image capturing device 30 and a size of a detection region of the cell detection chip to be detected (step S302), wherein the detection region includes a plurality of marks for positioning the images.

Specifically, when the image detection system 2 controls the image capturing device 30, to reduce the number of images during the scanning of the entire chip, in an embodiment of the invention, image scan is performed using a 4× objective lens. The image resolution achieved by the 4× objective lens is about 0.62 pixels/μm, and calculating based on an average size of 10 μm of white blood cells, the resolution that one cell can be assigned is about 6*6 pixels, and this resolution is sufficient to identify cell fluorescence and perform basic determination of circulating tumor cells.

It should be mentioned that, in the above embodiments, the image detection system 2 controls the image capturing device 30 to move using the electronic device 20 and records the images via a recording method. In another embodiment, the image detection system 2 can be made into a portable small electronic control platform suitable for any microscope system. The image detection system 2 is placed on the platform of the optical microscope itself, the microscope platform is moved for the most basic position calibration, and the chip center is moved to the middle of the objective lens.

Moreover, since a high-density cell detection method is used in the cell detection chip of the present embodiment, the difference between images of different regions is less significant. To increase the difference to facilitate subsequent image stitching, in the present embodiment, marks such as numbers, alphabets, marks, and patterns are increased at the bottom of the cell detection chip, and the edges of the marks are strengthened by using an optical dark field effect as needed. In particular, the principle of optical dark field is blocking incident light by using an opaque structure such that light cannot directly enter the objective lens and eyepiece. In the absence of an object, the vision is completely dark, and in the presence of an object, light is diffused at the edge of the object such that the edge thereof becomes bright and visible in the dark. Accordingly, simple identification and positioning can be performed on images in different regions without interfering with the fluorescent signal.

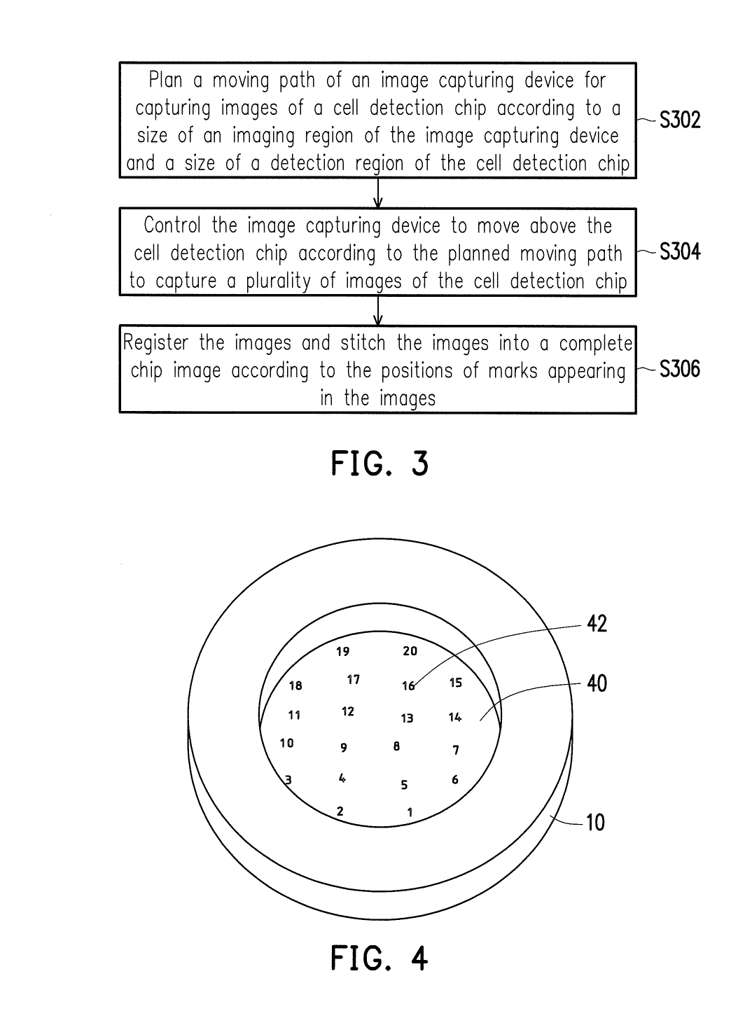

FIG. 4 is a schematic diagram illustrating the detection region of a cell detection chip according to an embodiment of the invention. Referring to FIG. 4, in the present embodiment, the detailed structure in the detection region 40 of the cell detection chip 10 in FIG. 1 is shown. In particular, the detection region 40 includes numeric patterns 42 arranged in a zigzag form, and the numeric patterns 42 are, for instance, made by using a photoresist, and numeric patterns 42 having a more complex shape are used as the marks of each of the regions in the detection region 40 and used by the electronic device 20 as reference for subsequent image stitching. Moreover, the corresponding positions of the captured images on the cell detection chip can be obtained via the size of the numbers using the method.

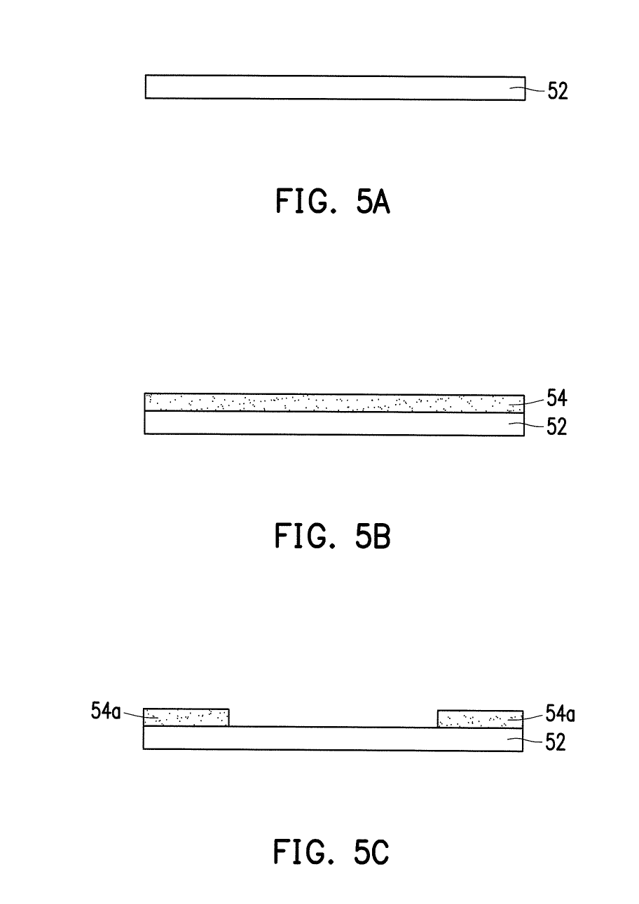

It should be mentioned that, in the production of the photoresist, in an embodiment of the invention, the microstructure on the slide is produced using the photoresist of an SU-8 3000 system, and then the numeric patterns on the slide are produced using the photoresist of AZ 9620. The production process thereof is described below with reference to FIGS. 5A to 5E:

In FIG. 5A, the surface of the slide 52 is washed using acetone, isopropanol, and DI water in order, and then the slide 52 is placed on a heating plate for baking to remove moisture (120° C., 5 minutes).

In FIG. 5B, the slide 52 is moved to a spin coating machine for coating a photoresist 54. In particular, the photoresist 54 used in the present embodiment is SU-8 3010. After the coating is complete, the step of soft bake is performed to remove most of the solvent in the photoresist 54 to make the structure more stable. After the temperature of the slide 52 is reduced to room temperature, exposure is performed on a single side. After exposure, post-exposure baking is performed to strengthen the structure producing a reaction during exposure. Upon completion, natural cooling is performed again.

In FIG. 5C, the photoresist 54 on the slide 52 is developed using the developing solution of SU-8 to generate a structure. To confirm whether the structure is complete, after developing for 30 seconds, rinsing can be performed by isopropanol, and whether the development is complete is confirmed after drying. After the development is complete, the isopropanol on the slide 52 is washed using DI water to complete the produce of a microstructure 54a of the runner.

It should be mentioned that, since SU-8 has limited adhesion to glass, a smaller mark cannot be attached to the glass surface, and therefore the marks are produced using an AZ 9260 photoresist. Before the AZ 9260 photoresist is coated, the slide 52 is first washed using isopropanol and DI water in order, and after baking is performed to remove moisture, evaporation is performed for 5 minutes using HMDS to increase the adhesion of the AZ 9260 photoresist on the slide 52.

In FIG. 5D, an AZ 9260 photoresist 56 is coated on the slide 52 on which the microstructure 54a is formed in a thickness of 10 micrometers under the parameters of 2000 rpm and 30 seconds. Then, the slide 52 is placed on a heating plate at 100° C. for soft bake for about 2 minutes. After the soft bake, exposure is performed, and the dose of the exposure is 200 mJ/cm².

In FIG. 5E, a developing solution of AZ 400K is mixed with DI water (in a ratio of 1:3), and the development time is about 90 seconds. The slide 52 on which the AZ 9260 photoresist 56 is coated is developed, and after development, the slide 52 is washed using DI water and placed on a heating plate for hard bake (120° C., 5 minutes) to complete the produce of the microstructure 56a of the marks.

Returning to the process of FIG. 3, the processor 26 then makes the control module 244 control the image capturing device 30 to move above the cell detection chip according to the moving path planned by the moving path planning module 242 to capture a plurality of images of the cell detection chip (step S304). Specifically, the image detection system 2 of the present embodiment can further allow the electronic device 20 to design functions such as moving speed and moving path. The design of the path can use the area of the imaging region of the image capturing device 30 as the basis for reference. For instance, measurement is performed by using a pattern (such as a grid of a cell counting plate, wherein one small cell is 50 micrometers) having a known size to obtain the imaging region of the image capturing device 30 at a certain magnification. If the imaging region of the image capturing device 30 is about 2 micrometers long and 1 micrometer wide, then the image capturing device 30 needs to move at least 7 times in the y-axis (based on a hole of 7 micrometers of the chip).

Lastly, the processor 26 executes the image stitching module 246 to position and stitch the images into a complete chip image according to the positions of the marks appearing in the images captured by the image capturing device 30 (step S306). Specifically, in an embodiment, the moving path planning module 242, for instance, further decides the number and position of the marks in the cell detection chip according to the size of the imaging region of the image capturing device 30 and the size of the detection region of the cell detection chip such that each of the images of the image capturing device 30 includes at least one mark. Accordingly, the image stitching module 246 can obtain the corresponding positions of the marks in the chip images based on the marks in each of the images, and stitching is performed on adjacent images by using the patterns of the marks at this point to obtain a complete chip image. In another embodiment, the image stitching module 246 can directly find the adjacent images according to the order of image capture or the position of image capture in the moving path of each of the images and stitching the adjacent images according to the positions of the marks appearing in the adjacent images to obtain the complete chip image.

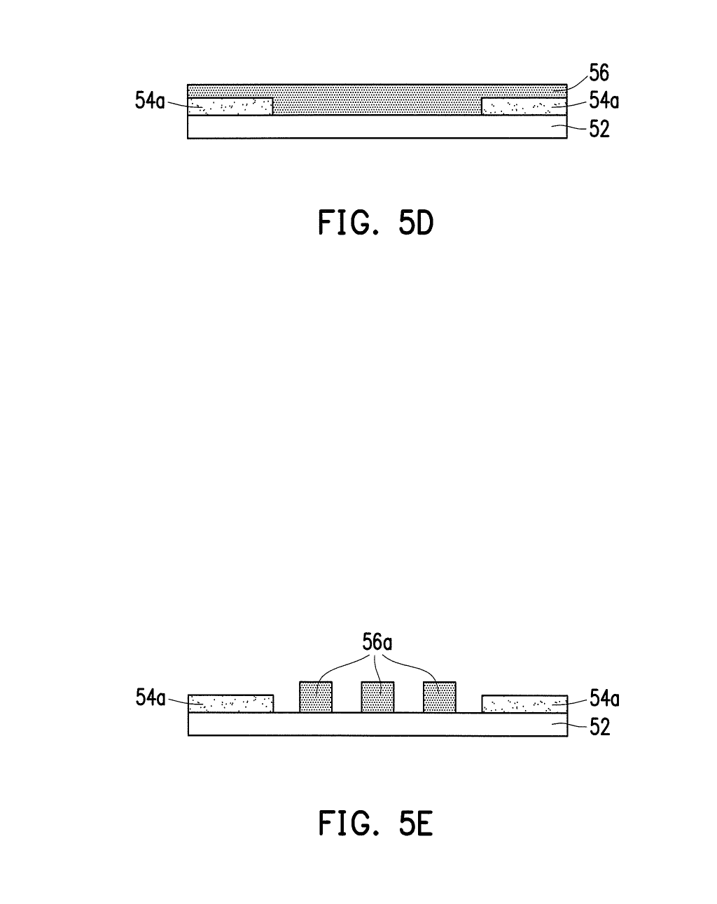

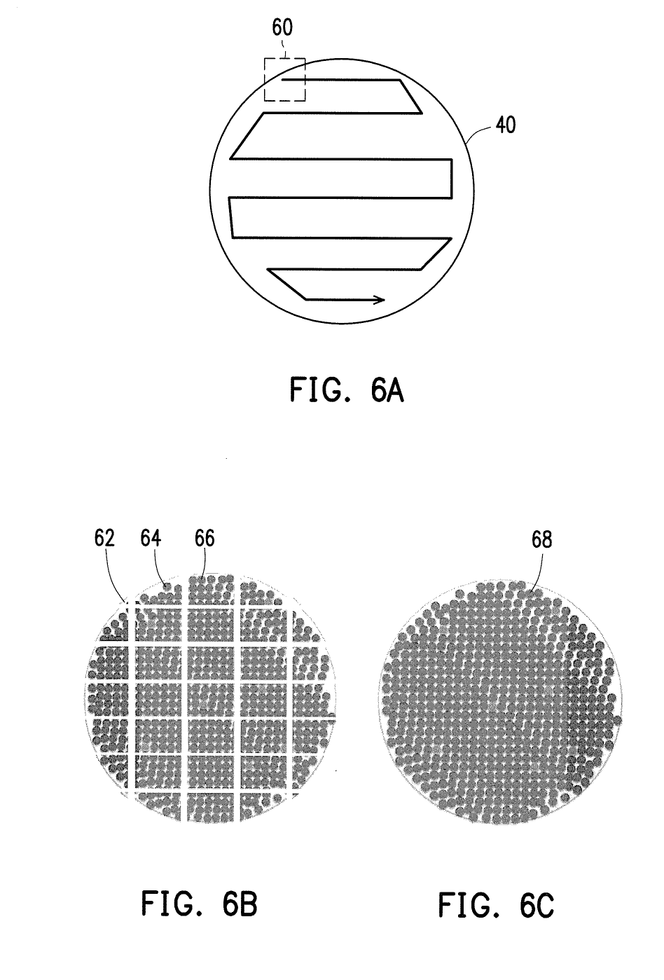

For instance, FIG. 6A is a schematic diagram illustrating the movement of an image capturing device according to an embodiment of the invention. FIG. 6B and FIG. 6C are schematic diagrams illustrating image stitching according to an embodiment of the invention. Referring to FIG. 6A, the moving path of the image capturing device of the present embodiment is, for instance, a simple bow shape, and the image in each of the regions in the detection region 40 can be obtained by performing recording and timing capture on the imaging region 60. The maximum value of the movement speed of the image capturing device is, for instance, 0.35 mm/s, and to prevent the generation of blurring during recording, the movement speed of the image capturing device can be set to 0.25 mm/s. FIG. 6 shows the images captured when the image capturing device is moving (such as images 62, 64, and 66). By stitching these images, a complete chip image 68 as shown in FIG. 6C can be obtained.

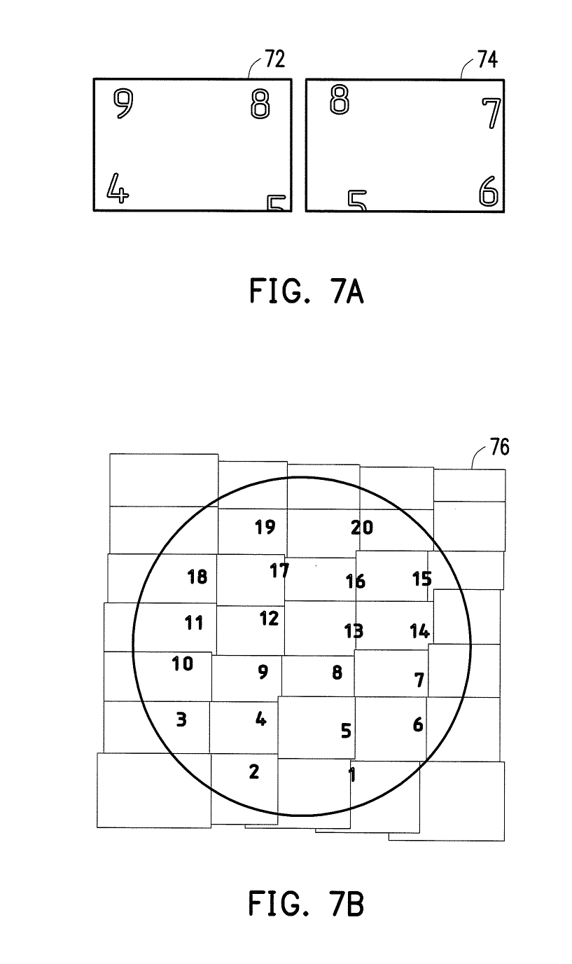

FIG. 7A and FIG. 7B are schematic diagrams illustrating image stitching according to the positions of marks in the images according to an embodiment of the invention. Referring to FIG. 7A, in the present embodiment, since each of the regions in the detection region of the cell detection chip has a corresponding numeric pattern and the images captured by the image capturing device in the image capturing process are overlapped, the images of these regions can be stitched into a complete chip image via an image processing software using the characteristics of the numeric patterns appearing in the overlapped portions. For instance, an image 72 and an image 74 can be stitched by the characteristics of a numeric pattern 8 and a numeric pattern 5 appearing in the image 72 and the image 74. The adjacent images are stitched one by one via the above method to obtain a complete chip image 76 shown in FIG. 7B.

Based on the above, in the image positioning and stitching method and the image detection system of a cell detection chip of the invention, images of each of the regions in a detection region of the cell detection chip which are disposed with marks for positioning, are captured by a camera in a manner of recording and timing capture. Next, via an image stitching algorithm, the marks in each of the images and images having the same marks are found, and then each of the images is positioned and stitched according to the positions of the marks in the images such that the marks in the images are overlapped after stitching. Via the assistance of the marks, the electronic device can rapidly identify the overlapped portions and stitch the chip images into a complete chip image.

Although the disclosure has been disclosed by the above embodiments, they are not intended to limit the disclosure. It is apparent to one of ordinary skill in the art that modifications and variations to the disclosure may be made without departing from the spirit and scope of the disclosure. Accordingly, the protection scope of the disclosure will be defined by the appended claims.

What is claimed is:

1. An image positioning and stitching method of a cell detection chip suitable for an electronic device having a processor to control an image capturing device to capture images of the cell detection chip and position and stitch the captured images, the method comprising the following steps:
planning a moving path of the image capturing device for capturing the images of the cell detection chip according to a size of an imaging region of the image capturing device and a size of a detection region of the cell detection chip, wherein a plurality of marks for positioning the images is disposed in the detection region;
controlling the image capturing device to move above the cell detection chip according to the planned moving path to capture a plurality of images of the cell detection chip; and
positioning the images and stitching the images into a complete chip image according to positions of the marks appearing in the images.

2. The method of claim 1, wherein the step of positioning the images and stitching the images into a complete chip image according to positions of the marks appearing in the images further comprises:
finding adjacent images according to an order of image capture or a position of image capture in the moving path of each of the images; and
stitching the adjacent images according to the positions of the marks appearing in the adjacent images to obtain the complete chip image.

3. The method of claim 1, wherein a number and a position of the marks in the cell detection chip comprise deciding according to a size of the imaging region of the image capturing device and a size of the detection region of the cell detection chip such that each of the images captured by the image capturing device comprises at least one of the marks.

4. The method of claim 1, further comprising:
producing the marks using a photoresist on the cell detection chip, wherein the photoresist comprises SU-8 or AZ9260.

5. The method of claim 4, wherein the step of producing the marks using the photoresist on the cell detection chip comprises:
coating a first photoresist on a slide surface and performing soft bake, exposure, and development to produce a microstructure of a runner; and
coating a second photoresist at the position of each of the marks on the slide surface and performing soft bake, exposure, and development to produce a microstructure of the mark;

6. The method of claim 1, wherein the marks comprise one of numbers, alphabets, signs, and patterns or a combination thereof.

7. An image detection system of a cell detection chip, comprising:
an image capturing device, disposed above the cell detection chip, moving to capture a plurality of images of the cell detection chip; and
an electronic device, comprising:
a connection device, connected to the image capturing device;
a storage device, storing a plurality of modules; and
a processor, coupled to the connection device and the storage device, executing the modules stored in the storage device, the modules comprising:
a moving path planning module, planning a moving path of the image capturing device for capturing the images of the cell detection chip according to a size of an imaging region of the image capturing device and a size of a detection region of the cell detection chip, wherein a plurality of marks for positioning the images are disposed in the detection region;
a control module, controlling the image capturing device to move above the cell detection chip according to the moving path planned by the moving path planning module to capture the images; and
an image stitching module, positioning the images and stitching the images into a complete chip image according to positions of the marks appearing in the images.

8. The system of claim 7, wherein the image stitching module comprises finding adjacent images according to an order of image capture or a position of image capture in the moving path of each of the images and stitching the adjacent images according to the positions of the marks appearing in the adjacent images to obtain the complete chip image.

9. The system of claim 7, wherein the moving path planning module further decides a number and a position of the marks in the cell detection chip according to the size of the imaging region and the size of the detection region of the cell detection chip of the image capturing device such that each of the images captured by the image capturing device comprises at least one of the marks.

10. The system of claim 7, wherein the marks comprise one of numbers, alphabets, signs, and patterns or a combination thereof.
